(19) United States
(12) Patent Application Publication
(10) Pub. No.: US 2010/0172945 A1
(43) Pub. Date: Jul. 8, 2010

(54) EXTRUDED DELIVERY SYSTEM

(75) Inventors: Christopher Gregson, Princeton, NJ (US); Matthew Sillick, Hamilton, NJ (US)

Correspondence Address: WINSTON & STRAWN LLP;PATENT DEPARTMENT, 1700 K STREET, N.W., WASHINGTON, DC, 20006, US

(73) Assignee: FIRMENICH SA, GENEVA 8, (CH)

(21) Appl. No.: 12/663,102

(22) PCT Filed: Jun. 13, 2008
(86) PCT No.: PCT/IB08/52335

§ 371 Date: Dec. 4, 2009

Related U.S. Application Data
(60) Provisional application No. 60/944,927, filed on Jun. 19, 2007.

(51) Int. Cl. A61K 8/04; A61K 9/10; A23L 1/00; A61Q 11/00

(52) U.S. Cl. 424/401; 424/484; 426/89; 424/49; 424/488

(57) ABSTRACT

A solid delivery system for an oily active ingredient, comprises an extrudate of a melt-emulsion wherein the continuous phase of the emulsion comprises a matrix material and the dispersed phase comprises an oily active material and an effective amount of a viscosity modifying ingredient such as ethyl cellulose.

### TECHNICAL FIELD

The present invention relates to a delivery system in the form of an extrudate, particularly for ingredients sensitive to oxidation and/or thermal degradation. It also relates to a process for preparing such a delivery system.

### BACKGROUND AND PRIOR ART

Delivery systems or encapsulation systems are used in various industries to protect active ingredients. For instance, in the food industry they are often used to protect flavours, in particular against losses of volatile components (i) during storage prior to incorporation into the food products, (ii) during mixing of the flavor component with the other food ingredients, (iii) during food processing, such as cooking and baking, (iv) during transportation and storage and (v) during the preparation of the food product by the end-consumer.

Similarly, in the nutraceutical industry, they are often used to protect oxygen-sensitive active material, such as fish oils rich in polyunsaturated fatty acids, by providing an oxygen barrier around the material.

Due to the importance of the delivery system in such fields, it is not surprising that various different types of delivery system exist. Among the different systems known in the art, extrusion methods typically rely on the use of carbohydrate matrix materials which are heated to a molten state and combined with the active ingredient(s), such as an oxygen sensitive oil, before extruding and quenching the extruded mass to form a glass which protects the active ingredient(s).

One significant example of the prior art disclosure in this field is in the U.S. Pat. No. 3,704,137 which describes an essential oil composition formed by mixing oil with an antioxidant, separately mixing water, sucrose and hydrolyzed cereal solids with DE below 20, emulsifying the two mixtures together, extruding the resulting mixture in the form of rods into a solvent, removing the excess solvent and finally, adding an anti-caking agent.

Further examples are described in U.S. Pat. No. 4,610,890 and U.S. Pat. No. 4,707,367 in which compositions are prepared by forming an aqueous solution containing a sugar, a starch hydrolysate and an emulsifier. An essential oil is blended with the aqueous solution in a closed vessel under controlled pressure to form a homogeneous melt, which is then extruded into a relatively cold solvent, dried and combined with an anti-caking agent.

More recently, it is proposed in WO-A-2006/067647 to encapsulate an oil rich in polyunsaturated fatty acids by emulsifying the oil in a concentrated syrup of water and a carbohydrate material, extruding the emulsion through a die, cooling the extrudate and washing the solid extruded material with a solvent liquid to ensure removal of excess oil which has not been encapsulated. This provides an effective system for protecting the oil against oxidation.

The above-mentioned patents, and all the other prior art cited therein, are merely illustrative of the considerable volume of patent literature related to the fixation of active in various matrices and which, in essence, discloses the encapsulation by extrusion of active materials in glass-like polymeric materials, in particular carbohydrate matrices.

Thus, there is a clear and ongoing need across many industries to have access to delivery systems in the form of extrudates. Accordingly, it is an objective of the present invention to address this need.

However, in many matrix-forming extrusion processes described in the literature, the mixtures must often undergo lengthy residence times at high temperatures, particularly during the emulsification and extrusion stages. This is undesirable when the material to be encapsulated is sensitive to oxidation or is thermally labile since such active materials are rendered yet more sensitive by the increased exposure at these higher temperatures. A further problem which can manifest itself during extrusion is that unwanted reactions between matrix (encapsulating) ingredients and the active material are increased. This can be partially addressed by incorporating additional intermediate cooling steps, but this leads to increased processing time, manufacturing cost and overall complexity.

Other problems include inadequate loading of oily active ingredients in the matrix, droplet sizes which can only be of a reduced size by shearing thus raising the temperature during processing, and the inability to maintain the droplet size once formed.

It would, therefore, be desirable to address one or more of these issues.

### DESCRIPTION OF THE INVENTION

Thus, according to the present invention there is provided a solid delivery system for an oily active ingredient, comprising an extrudate of a melt-emulsion wherein the continuous phase of the emulsion comprises a matrix material and the dispersed phase comprises an oily active material and an effective amount of a viscosity modifying ingredient.

The delivery systems of the invention provide uniform distribution of droplets of the oily active material in the matrix, thus stabilizing the active during storage and use. The active material is in fact retained throughout the matrix in a homogeneously dispersed manner such that release of the active material is balanced and gradual.

The delivery systems of the invention do not require special equipment and can be prepared using any current extruder typically used according to prior known “wet extrusion” or “dry blend” (also called “flash-flow”) techniques, the latter requiring feeding of a melt of an originally mainly solid mass into the extruder, and the former requiring the extrusion of a mainly fluid mass melt resulting from the prior solution of the matrix in a suitable solvent.

More objects and aspects of the invention will become apparent from the detailed description hereafter.

By extrusion methods we mean here methods according to which, typically, the matrix components, the oily active material and, optionally a plasticizer and an emulsifier, in the form of a melt-emulsion, are forced through a die and then quenched to form the solid product containing the oily active material. The terms “melt” and “melt-emulsion” are used interchangeably in the text and both denote a liquid matrix as a continuous phase with hydrophobic particles dispersed therein as the dispersed phase.

As used herein, the term “particles” means both solid articles and liquid droplets.

The melt can be formed in any way known in the art. This includes the heating of matrix ingredients to a temperature which allows the formation of an homogeneous melt, for example in a single or twin screw extruder. An alternative example is the dissolution of matrix ingredients in a solvent, preferably water, followed by the removal of some or all of this solvent by evaporation.

As the matrix in the delivery systems of the invention, there can be used any sugar or sugar derivative which can be readily processed to form a dry extruded solid. Particular examples of suitable materials include those selected from the group consisting of sucrose, glucose, lactose, levulose, fructose, maltose, ribose, dextrose, isomalt, sorbitol, mannitol, xylitol, lactitol, maltitol, pentatol, arabinose, pentose, xylose, galactose, hydrogenated starch hydrolysates, maltodextrin, Stabilite ® (origin: SPI Polyols, USA), oligofructans, trehalose, agar, carrageenan, inulin, other gums, polydextrose and derivatives and mixtures thereof.

According to a preferred embodiment of the invention, the matrix comprises maltodextrin or mixtures of maltodextrin and at least one material selected from the group consisting of sucrose, glucose, lactose, levulose, maltose, fructose, isomalt, sorbitol, mannitol, xylitol, lactitol, maltitol, trehalose and hydrogenated starch hydrolysates. The maltodextrin preferably has a dextrose equivalent not above twenty (≦20 DE) and more preferably a DE of between 5 and 18.

The above-mentioned matrix compositions are hereby given by way of example and they are not to be interpreted as limiting the invention. Although polysaccharides are mentioned above as specific examples, it is clear that any material which is extrudable and currently used as a matrix material in the production of extruded solids is adequate for the purposes of the invention.

The oily material, also referred to as “active ingredient” can designate a single hydrophobic compound or a composition, such as flavours, fragrances, pharmaceuticals, nutraceuticals or other ingredients, which one wishes to encapsulate.

In conventional extrusion systems, the long residence time at high temperature during emulsification and/or extrusion can lead to unwanted reactions of the active material, particularly where the material is oxygen and or heat-sensitive. Degradation reactions include, for instance, transesterification of esters, formation of esters of carboxylic acids, oxidation of aldehydes to acids, acetal formation from ketones and aldehydes, isomerization of cis double bonds, polymerization and peroxidation of polyunsaturated fatty acids containing triglycerides, oxidation and rearrangement on sulphur-containing compounds, acid catalysed hydrolysis of matrix ingredients and thermally induced free radical reactions.

Nevertheless, the delivery system used in the present invention has been found to be particularly effective in protecting oxygen and/or thermally sensitive active materials. Therefore, it is particularly preferred that the active ingredient comprises one or more oxygen or thermally sensitive ingredient.

Preferably, the invention is advantageously employed for the encapsulation of volatile or labile flavouring, perfuming or nutraceutical ingredients or compositions, in particular hydrophobic liquids, which are soluble in organic solvents but only very weakly soluble in water. More particularly, the flavouring, perfuming or nutraceutical ingredient or composition encapsulated according to the invention is preferably characterised by a Hildebrand solubility parameter smaller than 30 [MPa]^{1/2}. The aqueous incompatibility of most oily liquids can be in fact expressed by means of Hildebrand's solubility parameter δ which is generally below 25 [MPa]^{1/2}, while for water the same parameter is of 48 [MPa]^{1/2}, and of 15-16 [MPa]^{1/2} for alkanes. This parameter provides a useful polarity scale correlated to the cohesive energy density of molecules. For spontaneous mixing to occur, the difference in δ of the molecules to be mixed must be kept to a minimum. The Handbook of Solubility Parameters (ed. A. F. M. Barton, CRC Press, Boca Raton, 1991) gives a list of δ values for many chemicals as well as recommended group contribution methods allowing to calculate δ values for complex chemical structures.

The terms “flavour or fragrance compound or composition” as used herein, are thus deemed to define a variety of flavour and fragrance materials of both natural and synthetic origin. They include single compounds and mixtures. Natural extracts can also be encapsulated in the extrudate; these include e.g. citrus extracts, such as lemon, orange, lime, grapefruit or mandarin oils, or essential oils of spices, amongst other. Particularly preferred active materials in this class for encapsulation are flavour compositions containing labile and reactive ingredients such as berry and dairy flavors.

Further specific examples of such flavour and perfume components may be found in the current literature, e.g. in Perfume and Flavour Chemicals, 1969, by S. Arctander, Montclair N.J. (USA) ; Fenaroli's Handbook of Flavour Ingredients, CRC Press or Synthetic Food Adjuncts by M. B. Jacobs, van Nostrand Co., Inc. They are well-known to the person skilled in the art of perfuming, flavouring and/or aromatizing consumer products, i.e. of imparting an odour or taste to a consumer product.

An important class of oxygen-sensitive active materials that can be encapsulated in the delivery system of the present invention are “oils rich in polyunsaturated fatty acids”, also referred to herein as “oils rich in PUFA's”. These include, but are not limited to, oils of any different origins such as fish or algae. It is also possible that these oils are enriched via different methods such as molecular distillation, a process through which the concentration of selected fatty acids may be increased. Particularly preferred compositions for encapsulation are nutraceutical compositions containing polyunsaturated fatty acids and esters thereof.

Specific oils rich in PUFA's for use in the present delivery system include eicosapentanoic acid (EPA), docosahexanoic acid (DHA), arachidonic acid (ARA), and a mixture of at least two thereof.

Such oils may, optionally, be supplemented with an antioxidant. For example, the antioxidant-supplemented oil may comprise added ascorbic acid (vitamin C) and/or tocopherol (vitamin E). Tocopherol may be α-, γ-, or δ-tocopherol, or mixtures including two or more of these, and is commercially available. Tocopherols are soluble in oils and may be easily added at amounts in the range of 0.05-2%, preferably 0.1-0.9%, of the supplemented oil comprising the antioxidant.

The oily active material is preferably present in the extrudate in a concentration ranging from about 5% to about 40% by weight, based on the total weight of the extruded delivery system.

In the context of the present invention, the viscosity modifier is any material able to significantly increase effective viscosity of the oily phase at the temperature of the process. In particular, suitable viscosity modifiers are those which increase the viscosity of the oil to at least 10 times more, more preferably to at least 100 times more than the unviscosified oil. Examples of suitable viscosity modifiers include ethyl cellulose (e.g. the Ethocel range from Dow Chemicals), hydrophobic silicas, silicone oils, high viscosity triglycerides, sucrose esters of fatty acids, polyglycerol esters of fatty acids, organophilic clay, oil soluble polymers, high viscosity mineral oil (paraffinic and naphthenic liquid hydrocarbons), oleum treated and hydrogenated mineral oils, petroleum jelly, microcrystalline waxes and paraffin waxes.

The preferred viscosity modifier is ethyl cellulose since it is found to provide the additional advantage of having surface active properties that lower the interfacial tension between the oily active and the matrix, thereby lowering yet further the energy required to form an emulsion.

Preferably, the molecular weight of the ethyl cellulose is preferably within the range of from 50,000 to 2,000,000, more preferably from 75,000 to 1,500,000, most preferably from 100,000 to 1,250,000.

Preferably, the viscosity of the modified cellulose ether is from 50 mPa·s to 1,000 mPa·s, more preferably 75 mPa·s to 750 mPa·s, most preferably 100 mPa·s to 500 mPa·s, measured as a 5% solution based on 80% toluene 20% ethanol, at 25° C. in an Ubbelohde viscometer.

The amount of viscosity modifier present in the dispersed oily phase depends on the nature of the viscosity modifier and the oily active material and can be adjusted accordingly by the skilled person to achieve the correct viscosity. Nevertheless, it has been found that excellent results are achieved if, when the viscosity modifier is ethyl cellulose, the weight ratio of oily active material to ethyl cellulose is from 200:1 to 1:4, more preferably 50:1 to 1:2, even more preferably 40:1 to 1:1, most preferably 30:1 to 2:1, e.g. from 20:1 to 5:1.

It may be desirable to include one or more additional ingredients to increase the solubility or dispersibility of the viscosity modifier.

Optionally and advantageously, an emulsifier may be added to the mixture. This is found to decrease the interfacial tension between the oil and melt phases thereby lowering the energy for droplet formation. Additionally, it can stabilize the droplets once formed. Examples of suitable emulsifiers include lecithin, modified lecithins such as lyso-phospholipids, DATEM, mono-diglycerides of fatty acids, sucrose esters of fatty acids, OSA starch, sodium octenyl succinate modified starch, gum Arabic, citric acid esters of fatty acids, and other suitable emulsifiers as cited in reference texts such as Food Emulsifiers And Their Applications, 1997, edited by G. L. Hasenhuettl and R. W. Hartel.

Lecithins and modified lecithins are particularly preferred emulsifiers for use in the present invention. Suitable examples include, but are not limited to soy lecithin (such as Yelkin S S, ex Archer Daniel Midlands) and lyso-phospholipids (such as Verolec HE60, ex Lasenor).

Other optional ingredients can be present in the matrix. For instance, water may be present to modify the characteristics of the carbohydrate. For example, for a carbohydrate glass having a DE (dextrose equivalent) of 18, from 5 to 15% of water in the mixture may be present in the final product.

Similarly, adjuvants such as food grade colorants can also be added in a generally known manner, to the extrudable mixtures of the invention so as to provide coloured delivery systems.

The delivery system in the form of the extrudate preferably comprises particles of substantially uniform granulometry. Preferably the average particle size, based on the mean diameter, of the granules is from 200 to 4000 microns. The extrudate can be formed into granules by a variety of processes, of which the most preferred are described below.

Where the encapsulated material comprises a flavour oil, it can be advantageously used to impart or modify the organoleptic properties of a great variety of edible products, i.e. foods, beverages, pharmaceuticals and the like. In a general manner, they enhance the typical organoleptic effect of the corresponding unextruded flavor material.

Where the active material is an oil rich in polyunsaturated fatty acids or a nutraceutical composition comprising such an oil, it can be provided in any foodstuff where health benefits are desired. In such products, a further advantage of the present delivery system is that it can mask the flavour of the oil rich in polyunsaturated fatty acids, which may not be compatible with the flavour of the foodstuff into which it is incorporated.

The concentrations in which the delivery system can be incorporated in such consumer products vary in a wide range of values, which are dependent on the nature of the consumer product and that of the particular delivery system of the invention used.

Typical concentrations, to be taken strictly by way of example, are comprised in a range of values as wide as from a few p.p.m. (parts per million) up to 5 or even 10% of the weight of the flavoring composition or finished consumer product into which they are included.

The extruded delivery system formed from the melt-emulsion of the matrix component(s), oily active material and viscosity modifier are produced by a method which is modified from the current extrusion methods known in the art in that the viscosity modifier is mixed into the dispersed oily phase prior to an emulsification step.

Thus, the invention also concerns a process for preparing a stable active ingredient delivery system in the form of an extrudate, which comprises the steps of:
a) mixing an oily active material with a viscosity modifier to provide a substantially homogeneous liquid having an increased viscosity compared to the oily active material;
b) mixing the liquid from step (a) with an extrudable matrix material;
c) emulsifying the mixture to produce an emulsion in which the continuous phase comprises the matrix material and the dispersed phase consists of the viscosity modifier and the oily active ingredient;
d) extruding the emulsified mass through a die;
e) optionally forming particles of the extruded material; and
f) optionally drying the extrudate.

Optionally and advantageously, an emulsifier may be added in step (a) and/or step (b).

According to a preferred embodiment of the process of the invention, step a) is carried out by high-shear mixing of the ingredients with heat, if necessary. Optionally a co-solvent or surfactant, e.g. lecithin, can be present. This reduces the energy required to form the substantially homogeneous dispersion or solution.

Step c) is preferably carried out using an in-line emulsification process. Due to the presence of the viscosity modifier in the dispersed phase, it is found that the energy and/or time required to achieve emulsification is significantly less than conventionally required in known processes. This in turn reduces the temperature increase during emulsification and/or allows for very short time exposure of the sensitive active material to high temperatures.

This is in contrast to various known processes, where an intermediate cooling step is typically required in order to cool down the melt emulsion and, to other known processes, where the melt emulsion needs to be held at high temperature for very long periods, e.g. up to an hour, during extrusion, leading to the problems highlighted above.

The reduced exposure of the active material treated according to the present invention also provides several additional advantages such as, in the case of flavours, a flavour profile closer to the original (unprocessed) flavour oil and, in the case of nutraceuticals such as oils rich in polyunsaturated fatty acids, less risk of degradation of the labile ingredients therein.

It is desirable that the dispersed phase has a viscosity close to that of the continuous phase. This is because it has been found that this can further reduce the energy input required during emulsification. Accordingly, the ratio of viscosity between the dispersed and continuous phases is preferably from 1:1,000 to 1:1, more preferably from 1:500 to 1:1, most preferably from 1:100 to 1:1 (most preferably it would be as close to 1:1 as possible) Excellent results have nevertheless been achieved within a ratio range of from 1:100 to 1:10.

Step d) is carried out under conditions which can be derived by the person skilled in the art through experimentation. For instance, temperatures between 90 and 130° C., pressures up to 100 bar (10⁷ Pa), and a die orifice having a predetermined diameter from about 0.250 to 10 mm can be used. Of course, different temperatures, pressures and diameters for the die are also possible and can easily be ascertained by the skilled person through unimaginative trial and error.

Similarly, the type and design of the equipment used requires no detailed description here, the expert in this field being well-acquainted with current apparatuses, their technical specificities and the choice of appropriate equipment for a desired specific shape and size of extruded solid.

Whilst the requirement of increased temperature in step d) is not desirable for the reasons given above, the advantage of the present invention is that the residence time in this step is sufficiently short that significant degradation or evaporation problems in the active ingredient typically associated with conventional extrusion methods can largely be avoided.

According to step e), the extrudate can be formed into particles by any suitable means. For instance, it can be chopped whilst it is still in a plastic state (melt granulation or wet granulation techniques), or it can be cooled in a liquid solvent to form the extruded solid, the shape and size of which can be adjusted as a function of the extrusion parameters before being ground, pulverised or the like.

If desired the die orifice itself can be equipped with a cutterknife or any other cutting device. Alternatively, the cutting device can be provided separately downstream from the die orifice.

According to step f), the particulate solid obtained by extrusion can be dried, for example by use of an anticaking agent such as silicon oxide.

Thus, the temperature and pressure conditions under which the extrusion step is carried out can therefore be adjusted by the skilled person without particular effort and as a function of the nature of the ingredients present in the emulsion and of the quality of the product which is desired to obtain, i.e. its granulometry and shape. By contrast, the conditions for forming the emulsion prior to extrusion are not known and are defined by the limits cited herein.

### Examples

The invention will now be described in further detail by way of the following examples.

### Example 1

### Preparation of an Extruded Delivery System

| Ingredient | grams |
|---|---|
| Maltodextrin 18 DE⁽¹⁾ | 1000 |
| Sucrose | 1000 |
| Water | 400 |
| Lecithin⁽²⁾ | 2 |
| Ethyl Cellulose⁽³⁾ | 20 |
| Oil Rich in PUFAs⁽⁴⁾ | 200 |

⁽¹⁾Star-Dri 18, ex Tate and Lyle, Inc
⁽²⁾Yelkin SS, ex Archer Daniel Midlands
⁽³⁾Ethyl Cellulose (Ethocel Standard 300), ex. Dow Chemical Company, USA
⁽⁴⁾Tuna Fish oil, ex Ocean Nutrition Company, Canada

Viscosified fish oil was prepared by heating it with the ethyl cellulose and lecithin under high shear mixing. Separately, the maltodextrin and sucrose were dissolved in water and heated to 126° C. to reduce the water content to approximately 7%. The viscosified fish oil mixture was added to the maltodextrin/sucrose mixture and mixed to form a uniform dispersion of oil droplets within a continuous melt phase of the maltodextrin/sucrose mixture. The mixture was extruded under 2 bar pressure through a die plate with 0.5 mm holes. The extrudate was dried and 1% silicon dioxide was added as a free flow agent. The final product contained 10% fish oil by weight, 3.9% moisture and had a glass transition temperature of 35.7° C.

### Example 2

Experiment 1 was repeated but instead of fish oil, orange oil (ex Firmenich, Switzerland, ref 381106 08403TA) was used. The final product contained 10% orange oil by weight, 3.9% moisture and had a glass transition temperature of 35.7° C.

### Example 3

Experiment 1 was repeated but instead of 200 g of fish oil, 400 g were used. The final product contained 20% fish oil by weight, 3.9% moisture and had a glass transition temperature of 35.7° C.

### Example 4

Experiment 1 was repeated but instead of sucrose, isomalt was used. The final product contained 10% fish oil by weight, 4.2% moisture and had a glass transition temperature of 33.4° C.

### Example 5

Experiment 1 was repeated but instead of Ethocel 300, Ethocel 100 was used. The final product contained 10% fish oil by weight, 3.9% moisture and had a glass transition temperature of 35.7° C.

### Example 6

### Preparation of an Extruded Delivery System Using Twin-Screw Extrusion

| Ingredient | grams |
|---|---|
| Maltodextrin 18 DE⁽¹⁾ | 2000 |
| Water | 100 |
| DATEM⁽²⁾ | 40 |
| Triglyceride medium chains⁽³⁾ | 40 |
| Ethyl Cellulose⁽⁴⁾ | 20 |
| Orange Oil⁽⁵⁾ | 200 |

⁽¹⁾ex Roquette, France
⁽²⁾Di-Acetyl Tartrate Ester of Monoglyceride
⁽³⁾Neobee, ex AB Technology, U.K.
⁽⁴⁾Ethyl Cellulose (Ethocel Standard 300), ex Dow Chemical Company, USA
⁽⁵⁾ex Firmenich, Geneva, Switzerland ref. 381106 08403TA

Viscosified orange oil was prepared by heating it with ethyl cellulose under high shear mixing. A carbohydrate melt was formed using a twin screw extruder and the orange oil was injected by means of a gear pump into the molten mass where it was mixed to form a uniform dispersion of oil droplets within the continuous melt phase. The mixture was extruded under 2 bar pressure through a die plate with 2 mm holes. The final product contained 10% orange oil by weight, 3.9% moisture and had a glass transition temperature of 60° C.

### Example 7

### Evaluation of Ethyl Cellulose as Emulsion Aid

Emulsions having the following compositions were prepared:

TABLE 1

| Ingredient | Sample 1 (g) | Sample 2 (g) | Sample 3 (g) |
|---|---|---|---|
| Maltodextrin 18 DE⁽¹⁾ | 1000 | 1000 | 1000 |
| Sucrose⁽²⁾ | 1000 | 1000 | 1000 |
| Water | 500-550 | 500-550 | 500-550 |
| Peanut oil⁽³⁾ | 180 | 180 | 180 |
| Ethyl Cellulose⁽⁴⁾ | 20 | 20 | — |
| Lecithin⁽⁵⁾ | 1.875 | — | 1.885 |

⁽¹⁾Star-Dri 18, ex Tate and Lyle, Inc
⁽²⁾Granulated Pure Cane, ex Domino Foods, Inc
⁽³⁾LauAna brand
⁽⁴⁾Ethocel Standard 300, ex Dow Chemical Company, USA
⁽⁵⁾Yelkin SS, ex Archer Daniel Midlands.

In a first step, the sucrose and maltodextrin were dispersed and dissolved into the water under heat and agitation. The resulting solutions were then heated to reduce moisture content until a melt temperature of 128° C. was achieved. The mixture was then allowed to cool.

In a second step, oil blends comprising the oil, lecithin (if present) and ethyl cellulose (if present) were added to sucrose/maltodextrin solution and dispersed under shear until an emulsion had formed.

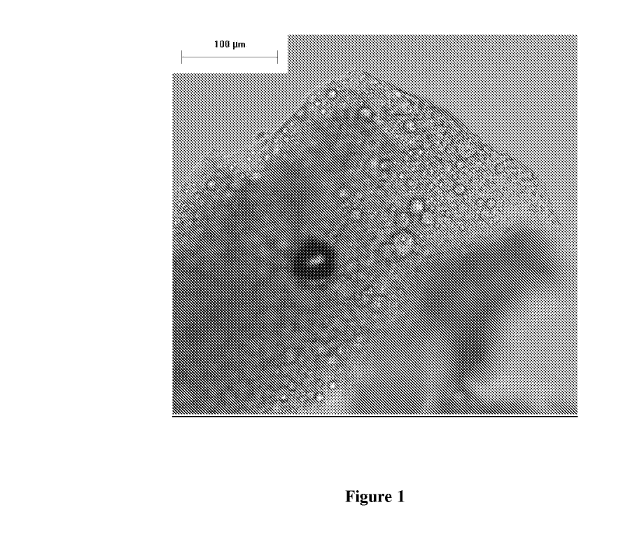

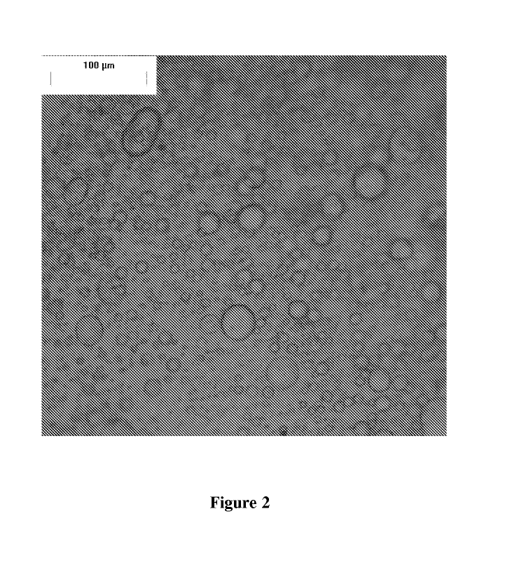

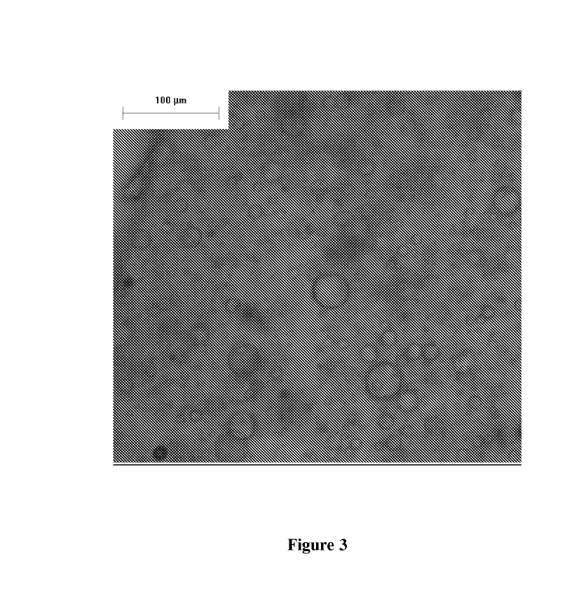

The melt-emulsion was allowed to cool and then examined by optical microscopy and photomicrographs. Samples 1 to 3 are shown in FIGS. 1 to 3 respectively.

Representative areas from the photomicrographs were analyzed with the software Image-Pro Plus Express (Media Cybernetics, Inc.). The diameter of each oil droplet present within the sample area was measured. The smallest droplets were found with the emulsion of sample 1, which had a mean oil droplet diameter of 3.5 μm. The emulsion of sample 2 had a mean droplet diameter of 5.4 μm. The emulsion of sample 3 had an average droplet diameter of 7.1 μm. Thus, the use of ethyl cellulose (sample 2) provides a smaller droplet size than obtained in the absence of ethyl cellulose (sample 3), and, in the presence of lecithin (sample 1), an even smaller droplet size was obtained demonstrating that desirable reduced droplet size can be obtained without increasing the energy (and heat) input.

1-16. (canceled)

17. A solid delivery system for an oily active ingredient, comprising an extrudate of a melt-emulsion having a continuous phase that comprises a matrix material, a dispersed phase that comprises an oily active material and an effective amount of a viscosity modifying ingredient, wherein the dispersed phase and continuous phase are present in a viscosity ratio of from 1:100 to 1:1.

18. The delivery system according to claim 17, wherein the dispersed phase has a viscosity that is at least 10 times greater than that of an identical dispersed phase in the absence of the viscosity modifier.

19. The delivery system according to claim 17, wherein the oily active material of the dispersed phase comprises an oil rich in polyunsaturated fatty acids.

20. The delivery system according to claim 17, wherein the viscosity modifying ingredient is a cellulose ether, a modified cellulose ether or a silicone oil.

21. The delivery system according to claim 20, wherein the viscosity modifying ingredient is ethyl cellulose.

22. The delivery system according to claim 21, wherein the oily active material and ethyl cellulose are present in a weight ratio of from 200:1 to 1:4.

23. The delivery system according to claim 17, wherein the matrix material is selected from the group consisting of sucrose, glucose, lactose, levulose, fructose, maltose, ribose, dextrose, isomalt, sorbitol, mannitol, xylitol, lactitol, maltitol, pentatol, arabinose, pentose, xylose, galactose, hydrogenated starch hydrolysates, maltodextrin, trehalose, oligofructans, agar, carrageenan, gums, polydextrose and mixtures thereof

24. The delivery system according to claim 23, wherein the matrix material is formed of maltodextrin or of a mixture of maltodextrin and at least one material selected from the group consisting of sucrose, glucose, lactose, levulose, maltose, fructose, isomalt, sorbitol, mannitol, xylitol, lactitol, maltitol and hydrogenated corn syrup.

25. The delivery system according to claim 17, wherein the oily active material is present in the extrudate in a concentration ranging from about 5% to about 40% by weight, based on the total weight of the delivery system.

26. A process for preparing a stable extruded active ingredient delivery system, which comprises:
mixing an oily active material with a viscosity modifier to provide a substantially homogeneous liquid having an increased viscosity compared to the oily active material;
mixing the substantially homogeneous liquid with an extrudable matrix material to form a mixture;
emulsifying the mixture to produce an emulsified mass that includes an emulsion having a continuous phase comprising the matrix material and a dispersed phase comprising the viscosity modifier and the oily active ingredient, with the dispersed phase and continuous phase being present in a viscosity ration of from 1:100 to 1:1;
extruding the emulsified mass through a die;
optionally forming particles of the extruded material; and
optionally drying the extrudate.

27. A stable extruded active ingredient delivery system formed by the process of claim 26.

28. A foodstuff, a beverage, an edible composition, a pharmaceutical composition, a nutraceutical composition, a chewing-gum or a toothpaste, comprising the delivery system according to claim 27.

29. A foodstuff, a beverage, an edible composition, a pharmaceutical composition, a nutraceutical composition, a chewing-gum or a toothpaste, comprising the delivery system according to claim 17.

30. A method of making a foodstuff, a beverage, an edible composition, a pharmaceutical composition, a nutraceutical composition, a chewing-gum or a toothpaste, which comprises:
forming a stable extruded active ingredient delivery system by the process of claim 26; and
adding the stable extruded active ingredient delivery system to a suitable composition to form a foodstuff, a beverage, an edible composition, a pharmaceutical composition, a nutraceutical composition, a chewing-gum or a toothpaste.
